(19) United States
(12) Patent Application Publication
(10) Pub. No.: US 2003/0199098 A1
(43) Pub. Date: Oct. 23, 2003

(54) Advanced tool for the quantification uniformity within biological processes and conversions

(76) Inventor: Oleg Kuznetsov, Providence, RI (US)

Correspondence Address: Oleg Kuznetsov, M.D., Suite B, 322 Veazie Street, Providence, RI, 02904-2100, US

(21) Appl. No.: 10/425,732

(22) Filed: Apr. 30, 2003

Related U.S. Application Data
(63) Continuation-in-part of application No. 09/495,371, filed on Feb. 1, 2000.

(51) Int. Cl. G01N 33/00

(52) U.S. Cl. 436/132

(57) ABSTRACT

Versatile method for the quantification of biological transformations has been developed to determine their interior regularity in a universal way. This life-aligned, multidimensional method demonstrates capability to identify an inner uniformity for the biological cycle beyond it perceptible manifestation. The improved capacity of the advanced Index-K quantification tool to adjust the baseline zero value for any preferred living cycle enables the tool to compute inward uniformity for virtually every biological processes or conversions.

Unlike other existing implements, the invented method is capable of reconciling the differences of meanings, simultaneously obtained on an idem subject by the various and disparate measuring methodologies, yet provides reasonably commensurable results to other exemplification, brought over diverse techniques, methods, technologies, or even units of measurement.

### CROSS-REFERENCES TO RELATED APPLICATION

I. Drug and Alcohol Abuse, Marc A. Schuckit, M. D., Third Edition, Chapter 3 Alcohol Metabolism, p.62-63, New York & London, 1989

II. Alcohol Alert, NIAAA, No. 35, PH731, Alcohol Metabolism, National Institute of Health, 1997

III. Eighth Special Report to the U.S. Congress on ALCOHOL and HEALTH from Secretary of Health and Human Services, September 1993

IV. U.S. Pat. No. 5,783,449, METHOD FOR QUANTIFYING ALCOHOL CATABOLISM, O. Kuznetsov, M. D., 1998

V. Nuclear Physics, by Enrico Fermi, Revised Edition, Chap. 1, p.1, p.42; Chap.3, p.111, The University of Chicago, Chicago & London, 1949/1974

VI. Nuclear and Particle Physics, W. S. C. Williams, Chap. 2.3, p.20-21, Clarendon Press, Oxford 1991

VII. Nuclear Physics, Alex E. S. Green, Chap. 6, p.196, New York & Toronto & London, 1955

VIII. Atomic Physics, Max Born, Eight Edition, Chap. X, ₇, p.334-335, New York, 1989

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

For nearly a hundred years we have constrained the law of nuclear physics to substitute a code of life in biological and pharmacological science. The omnipresent employment of the half-life postulation is accountable for impeding our quality of life.

The gravity of the situation does not decline when other estimation tools are used to compute the data, obtained by diversified methods, units of measurement or techniques. The problem remains unsolved: All these tools consistently fail to provide comparability of their results within a simple concurrency test. The comparison among various contingents makes little sense, following our inability to collate results from idem subject.

The instrument for quantifying inner uniformity of biological processes and conversions has been developed in response to inappropriate transference of the law of radioactive decay on biological science. The advanced Index-K tool not only brings convenience of data comparability obtained on the subject from diverse methodologies, but finally allows a fair comparison of results from different subjects, obtained by previously incommensurate technologies, methods, or even units of measurement as set forth in this specification.

### BRIEF SUMMARY OF THE INVENTION

The innovative Index-K quantification tool is designed to measure the factual regularity within biological processes and conversion.

While the quantitative variety of alive cyclic processes and conversions are nearly indefinite, the qualitative structure for all of them—where any living conditions must have beginning, culmination of development and end—is the same. Summarization of these qualitative features is obtainable on the quasi-parabolic graphical illustration of the cycle (FIG. 2), where space under ascending and descending shoulders considered to be a qualitative equivalent of energy released in opposed directions as a mingle effort to complete this biological cycle, following the law of nature when every action has equal and opposite reaction. Because the distinctive integrity of any biological process/conversion is a reflection of the unique equilibrium between these two forces, the advanced Index-K quantification tool has been established as the quotient of descending Area Under the Curve (AUC) over ascending AUC of the graphical representation for the preferred cycle, with an adaptable to zero baseline value (where required).

Unlike other tools, the contender not only allows us to compare the data simultaneously obtained from a subject by multiple methodologies, but finally permits the equitable comparison of results from different subjects, obtained by diverse techniques, units of measurement or methods. Considering the capability of the proposed instrument to measure inner uniformity for biological processes and conversion, the suggested use for it would be as follows:

To break the barrier of incomparability for interpreting biological data obtained from diverse measurement technologies.

To test, in a prompt and cost-effective way, a newly developed medication toward the presumed set of targets, without sacrificing safety requirements.

To predict scientifically (not statistically!) personal effectiveness in pharmacological treatment.

To determine the toxicity of drugs without killing a single creature.

To prevent the adverse or altered metabolic reactions from medication.

To measure the intensity of drug interactions.

To advise relatively “safe” alcohol consumption, based upon the state of our personal metabolic endurance.

To help diagnose alcohol disorder by its biological roots.

To detect diabetes type I or II, regardless of the highest value of blood sugar level, and far in advance of appearance of any clinical symptoms.

To reveal the personal vulnerability toward various deficiency or impaired metabolism disorders.

Rendering the optimum of ADMET (Absorption, Distribution, and Metabolism) efficiency for any biologically active matter.

Of course, the ultimate applications for this invention are not limited just to the examples described above; they are bounded only by our imaginations.

These objectives and advantages of present invention will become apparent to the readers after considering the following description and accompanying drawings.

### BRIEF DESCRIPTION OF SEVERAL VIEWS OF THE DRAWINGS

FIG. 1 graphical representation of the quantum mechanical nature for a disintegrative, proportionally-progressive transformation within the lifeless matter (alpha decay). It is ambiguously adapted by the pharmacology and biological science as an icon for the half-life computational tool.

FIG. 2 graphical representation of the quasi-parabolic changes, essentially present within every biological cycle of processes and conversions. It is used by the inventor as a foundation for the advanced Index-K computational tool.

FIG. 3 ethanol concurrency test (simultaneously performed on the same human subject during a single experimental session) in a graphical representation

1. three separate reading techniques from:

a. stripped skin,

b. non-stripped skin,

c. and breath;

2. obtained by detection from two dissimilar methods:

a. breath, and

b. skin biological responses;

3. measured by three different units of measurement:

a. mg/dl,

b. μA×45, and μA×40, as determined by functional capability of the measurement equipment.

FIG. 4 graphical illustration for a base line determination of the biological phases with non-zero (final, initial, or both) quantitative values of its indispensable quasi-parabolic alterations.

FIG. 5 graphical representation of an oscillation process within the glucose homeostasis, and the way for quantifying its inner normality by the advanced Index-K tool.

### DETAILED DESCRIPTION OF THE INVENTION

Because it is utterly impossible to display here all processes and conversions within the myriad variety of biological cycles, the inventor chiefly used for this purpose two key models which fully represent the whole eventual spectrum of their combinations, specifically: ethanol-related (bio-conversion with a common initial and final zero value) and human sugar metabolism (with the absent zero baseline of bio-conversion, as a radical alternative to the first basic model), where endogenous substance—glucose—is continually during lifetime being formed and utilized.

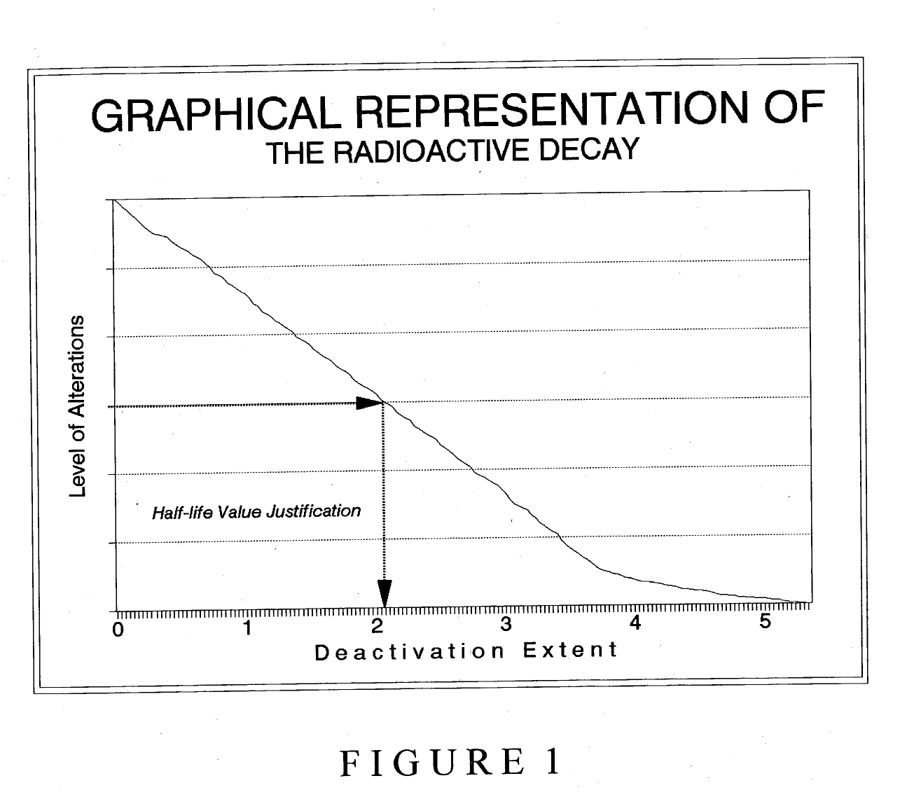

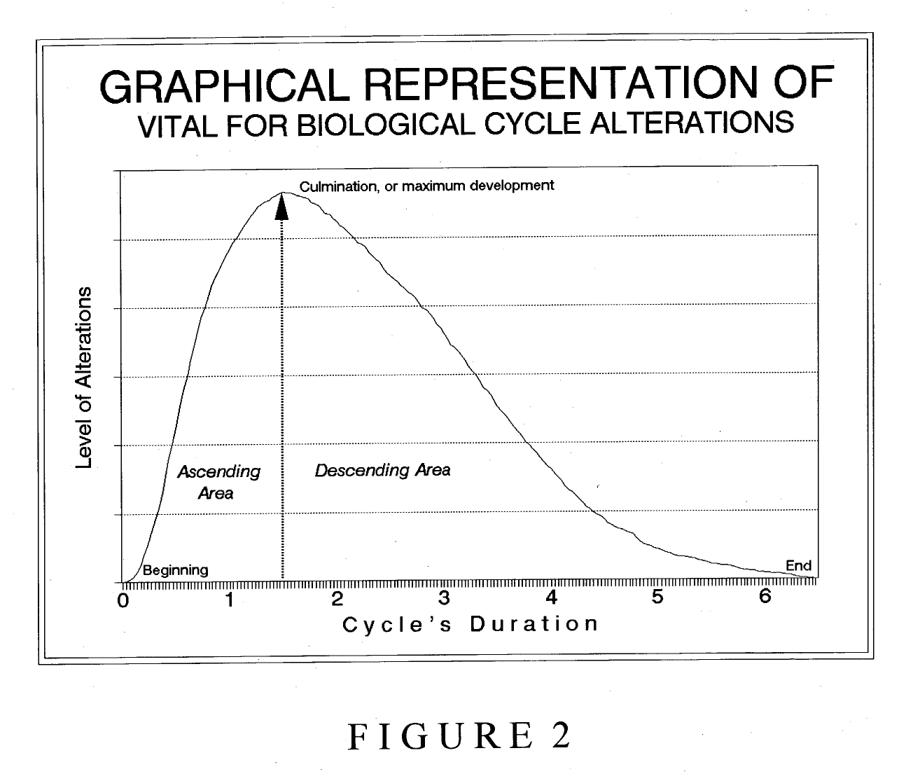

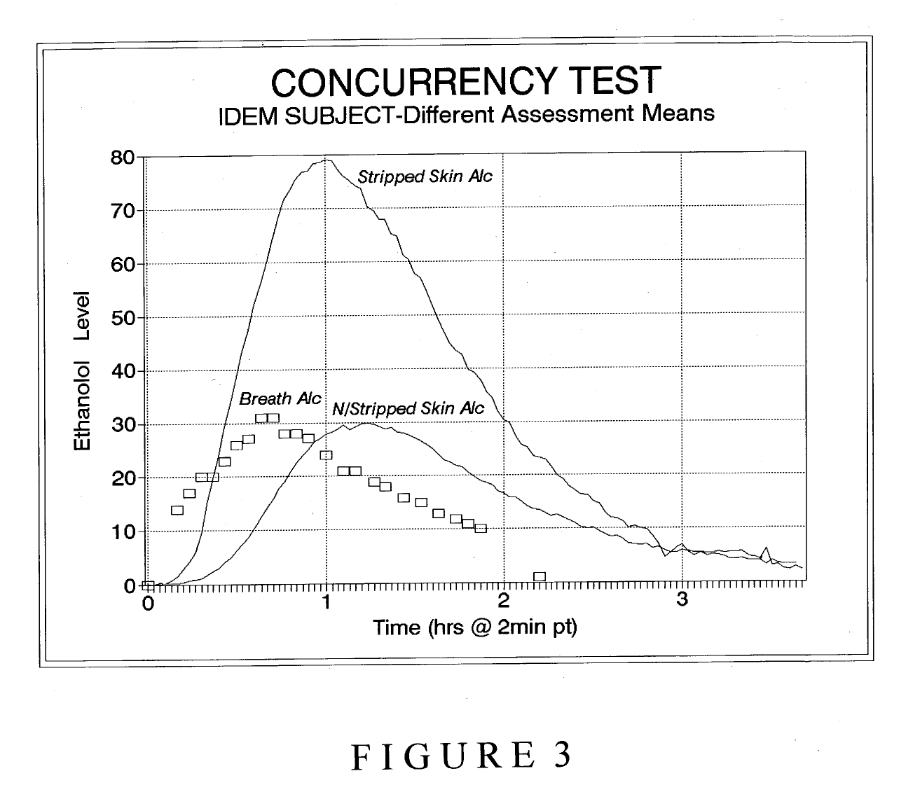

While the inanimate nature of the ubiquitous half-life theory assumes a logarithmic, proportionally-progressive disintegration process to be driven throughout time [VI, VIII], due to the collective parent-daughters radioactive chains of decay [V, VII] (see FIG. 1), living beings operate their processes via an asymmetrically charged parabolic fashion (see FIG. 2). This is why the common use of the “half-life” tool for clearance quantification of substances from the human body is inadequate. Even though the half amount of an introduced substance will be eliminated over a period of time, it is a glaring error to assume that this value presents genuine reciprocity within the organism, its tissues or organs. Living beings, due to their highly differentiated functional composition, conduct their processes and conversions in a multidirectional way, far more complex than lifeless matter does. The failure to apply the law of quantum mechanical nature on biological life becomes apparent from FIG. 3.

The following TABLE 1 of results describes the FIG. 3 curves:

| EQUIPMENT: | Intoximeter | Transcutaneous | Transcutaneous |
|---|---|---|---|
| | INTOX ER/IR | Ethanol Sensor (TES#39) | Ethanol Sensor (TES#33) |
| APPLIED TECHNIQUES: | breath samples | non-stripped skin | stripped skin |
| ESTIMATION | breath ethanol | excreted over the | excreted over the |
| METHODS FROM: | content | intact skin ethanol | stripped skin ethanol |
| UNITS OF | milligrams | microamperes | microamperes |
| MEASUREMENT: | per deciliter | multiplied by 45 | multiplied by 40 |
| DATA: | | | |
| Peak value | 31 (mg/dl) | 29.91 (μAx45) | 79.0 (μAx40) |
| Time to peak | 40 min | 74 min | 60 min |
| AUC (Area Under the Curve) | 2162.997 | 2883.615 | 6845.058 |
| Ascending part of AUC | 692 | 993.54 | 2301.63 |
| Descending part of AUC | 1346.997 | 1890.0076 | 4543.425 |
| Deactivation time | 132 min | ≧218 min | ≧220 min |
| Half-life | 89 min | 109 min | 127 min |
| Index-K | 1.95 | 1.9 | 1.97 |

The INTOXIMETER EC/IR 3000, which was used for the data presented in the TABLE 1, was made by Intoximeter, Inc. of St. Louis, Mo. The transcutaneous ethanol sensors (TES #33, #39) were made by Giner Inc. of Waltham, Mass.

While the breath estimation by 132 min indicates that the substance (ethanol) is gone from the bloodstream (deep lung breath samples are highly correlated ≈0.99 with blood alcohol concentration measured by gas chromatography), it still remains under biological conversion within other body tissues, particularly in the skin, for more than 220 min. The application of the half-life computation tool does not rectify this divergence. Moreover, it not only sustains the disparity of results for different tissues (89 min for blood, and over 127 min for skin), but also illustrates the total absurdity of its own meanings within the skin tissue exclusively (109 vs. 127 min)! Unfortunately, the half-life estimation tool yields separately incomparable results for different organs, tissues, and measurement techniques, precluding a true understanding of biological reciprocation. Equally poorly present themselves and the key compulsory assessments of bioavailability for contemporary pharmacokinetics of drugs: Time to peaks—40/74/60 min, and AUCs estimation of—2162.997/2883.615/6845.058. Meanwhile, for all three disunited techniques, different units of measurement, and disparate body tissues, the invented instrument reveals by the nearly identical value of Index-K—1.95/1.9/1.97—that the very same law of nature has driven this metabolic conversion. The highly commensurable results executed by the Index-K conspicuously surpass all other implements' values and confirm the internal uniformity of biological conversion for a chosen substance—ethanol in a given—31-year-old Caucasian male individual.

This simple concurrency test indisputably demonstrates that no single quantification tool available on the market can be trusted when initial measurements have been obtained from disparate body tissues, or by different units of measurement even on the same person. However, the gift of human body to subsist entirely depends on a wisely unified variety of diversified tissues. Therefore, common use of such tools restrains our knowledge of biological reciprocation to the constricted viewpoint with endless incomparability results within a single subject.

Nonetheless, ethanol is a drug with no different pharmacokinetic behavior than any other drug known to humankind. Even so-called controlled-release drugs, pharmacokinetic profiles, obey the same basic principle: Despite the gradually descending concentration after the peak it still retains a shape with asymmetrical shoulders, and initially-final zero value. The difference lies only in the half-life, bioavailability rates and other single or two-dimensional flawed estimation assumptions, but the illustration above persuasively shows that they are immaterial for the Index-K tool, based on a life-aligned, multidimensional foundation. However, alcohol metabolism is not limited just to a simple enzymatic oxidation of ethanol by the alcohol dehydrogenase [I, IV], but also involves other highly complicated bio-mechanisms [III] including the very same enzyme, cytochrome P450IIE1 (CYP2E1), which influences metabolism of the food, hormones, and medications [II]. These qualities make the model one of the best for consideration of any other preferred cycle of biological processes or conversions with a common initially-final quantitative zero value.

Dangerously, the half-life and bioavailability estimation tools are the established methods employed by pharmacology in determining the “proper” amount and “frequency” of drug administration, despite their complete inability to take into account the patient's metabolic response. In fact, the personal metabolic profile determines how an identical dose of drug, substance, or biologically active molecules produce various outcomes, ranging from complete indifference through expected effectiveness, to unwanted lethal effects. However, the individual's metabolic condition itself is not a biological constant and varies under numerous exogenous and endogenous conditions and, as the verification through a simple prism of concurrency test provided above shows, no single existing tool can credibly quantify it today. This is why nearly 2 million people die annually from what the FDA considers a safe dosage of medication.

Although alcoholism is caused only by the simple organic substance—ethanol—the measurement and counting of pharmacokinetic changes within an organism in the past 50 years have produced no single dependent variable for use as a biological marker for alcohol disorders. Currently in use is a long list of tools for counting ethanol, disposition during its metabolic conversion.

However, because all of them are derived from three basic models—the Widmark one-compartment open model with zero-order elimination kinetics; one compartment model with Michaelis-Menten kinetics; and one-compartment model with multiple Michaelis-Menten pathways—they are inherently prone to the same weakness, in spite of the variety of highly complex and sophisticated mathematical expressions. At this point there is no need to present all of them, because due to the strict two-dimensional nature (meanings versus time) they are unable to discern the cardinal individual differences in alcohol deactivation.

The described Index-K tool, by establishing the efficiency corridor up to 3.4 through the routine clinical experiments for alcohol catabolism on humans, allows us to discriminate between relatively “safe” and harmful alcohol consumption for any tested individual using similar methods and amount of ethanol administrations. This tool is applicable no matter which technique or equipment is used to obtain the initial readings. The clinical trials performed on 83 humans corroborated the ability of this tool to discriminate efficiency in alcohol catabolism between such human differences as the individual's stage of alcohol dependency and gender, even despite equivalent peaks of the blood alcohol level, with a statistical probability of 0.001. This was previously unthinkable in the substance abuse field.

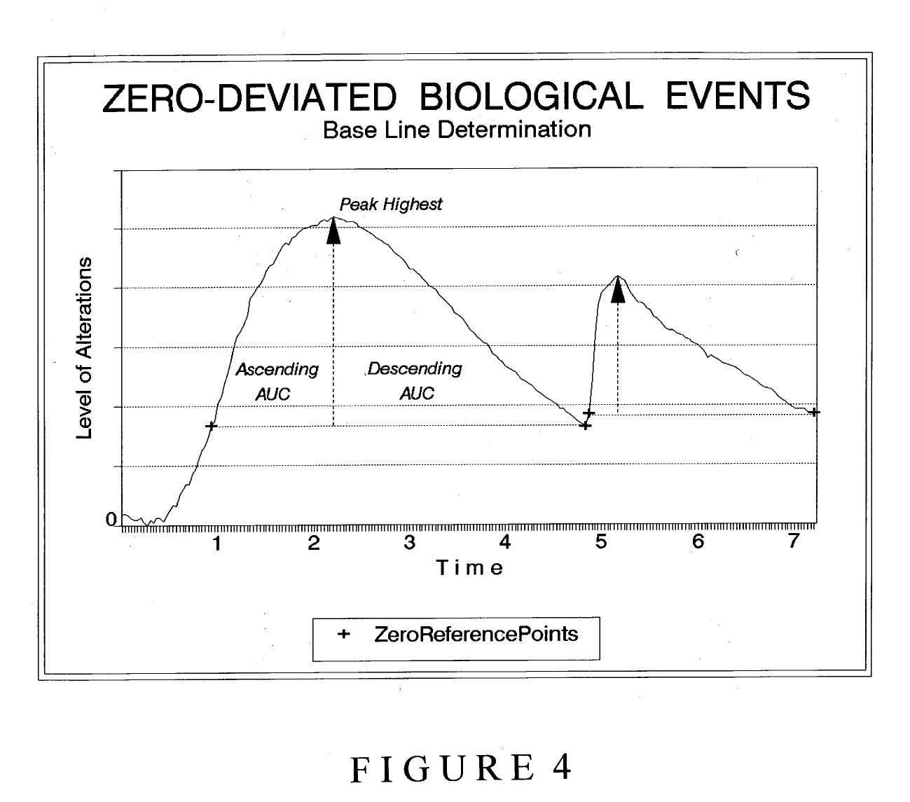

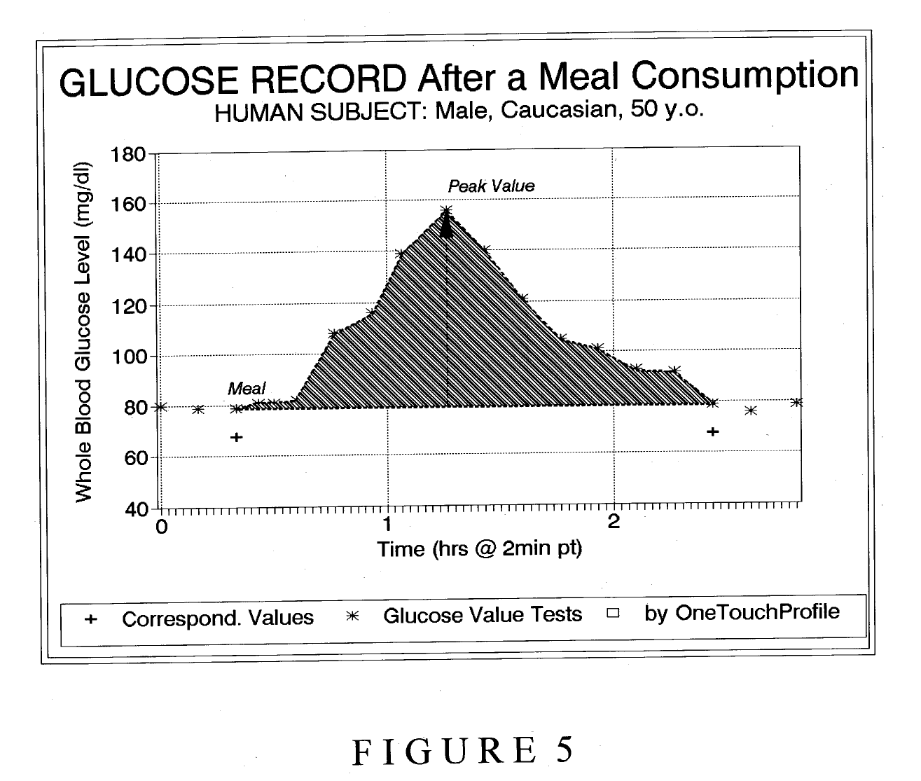

Nevertheless, applications for the Index-K tool have some limitations, where a biological cycle does not have an ideal closing or opening physiological zero value (see FIG. 4), required by the exacting ethanol-related model. In this case, an important modification was added to the Index-K tool to permit the withdrawal of any initial value, and replacing it with the zero-reference value, intended from the lowest primordial point of the ensuing quantitative changes corresponding with a final value for the chosen living cycle of process or conversion. FIG. 5 and TABLE 2, presented below, substantiate the capacity of the advanced Index-K tool to precisely compute inner regularity for just about any process and conversion, even with an entirely absent initially-final zero value of their physiological alterations.

The following TABLE 2 of results explains the FIG. 4

| EQUIPMENT: | Glucometer | |
|---|---|---|
| | One Touch Profile | |
| APPLIED TECHNIQUES: | small blood samples | |
| ESTIMATION | whole blood content | |
| METHODS FROM: | | |
| UNITS OF | milligrams | |
| MEASUREMENT: | per deciliter | |
| DATA: | | Zero-Reference Value: |
| Initially-final | 79 (mg/dl) | “0” |
| corresponding values | | |
| Peak value | 156 (mg/dl) | 156 − 79 = 77 |
| Time to peak | 56 min | |
| Integrated | 3810.01 | |
| AUC (Area Under the | | |
| Curve) | | |
| Ascending part of AUC | 1729 | |
| Descending part of AUC | 2081.01 | |
| Duration of the cycle | 126 min | |
| Half-life | Not quantifiable | |
| Index-K | 1.2035916 | |

To awake the oscillation process within an organism's homeostatic glucose conversion, meal consumption in equivalent of 50 g of carbohydrates within 5 minutes duration time has been applied.

The GLUCOMETER One Touch Profile, which has been used to acquire data presented in TABLE 2, was made by Johnson and Johnson Company, ©Lifespan Inc. of Milpitas, Calif.

Today's entire approach to prognostication of diabetes is mostly based on the sole measurement of elevated blood glucose levels as a reliable indicator of deficient insulin production by the beta cells. However, hyperglycemia is a common physiological condition and frequently occurs even among healthy people, for example, after consuming a meal (see FIG. 5) or under stressful situations. Such a hyperglycemic approach is incapable of revealing the latent forms of the disorder, or explaining away the 90% of patients who have non insulin-dependent (type II) diabetes. Rather than insulin deficiency, these diabetics produce two or three times the normal levels: Insulin just doesn't work for them efficiently. Moreover, this commonly accepted method fails to identify the earliest stages of diabetes, when the blood sugar level has not yet exceeded the upper limits of physiological norm on a permanent basis, but the disease has already begun. Another, much more precise method—the glucose tolerance test—startlingly employs the repeated measurements of blood sugar level within several hours after standardized meal consumption, but it fails to detect the earliest stages of diabetes as well, due to the unreliable estimation schemes applied for its quantification.

Although the discovery was initially made on an ethanol metabolism basis, it can also be extrapolated to determine the catabolic efficiency for virtually any measurable substance in the human body, since it has broken the code on which Mother Nature conducts all types of biological clearances. Because this tool is sufficient for detecting the slightest deviation in personal catabolism for the selected substance, the modification to this method described above allows us to detect a most minute irregularity in carbohydrate metabolism as well, something current methods cannot do. Establishing the efficiency corridor through routine clinical testing for sugar metabolism on humans, as it is done for alcohol catabolism [IV], will allow us to define diabetes type I or II regardless of borderline blood glucose level and well in advance of the appearance of any clinical symptoms. Conceivably, we might even be able to counteract the vicious effects of this currently incurable disease.

Because most drugs, as well as their therapeutic quantity, are alien and therefore harmful to a human body, and given the invention's capability to accurately quantify the catabolic property within the human body, another potent use for it would be in individually-tailored pharmacotherapy to pledge the patient's personal safety against the inappropriate dosage, and adverse or altered metabolic reactions to a medication. Sadly, quantification technologies currently in use limit our ability to predict such deadly side effects mostly to the use of statistics, and only when someone dies first do the fatal side effects become statistically reckonable. However, this can and should be avoid by application of the life-aligned, advanced Index-K quantification tool on pharmacokinetic data that already exist, or are being collected by pharmaceutical industry following standard common-sense rules and principals for clinical and experimental pharmacology trials. It is an especially valuable tool for the ongoing long-term treatments of chronic diseases such as diabetes, depression, asthma, schizophrenia, heart conditions, etc.

Although the advanced Index-K has been described above in considerable detail, it is to be understood that modifications may be made to the invention which do not exceed the scope of appended claims; modified forms of the present invention done by others skilled in the art to which the invention pertains will be considered infringements of this invention when those modified forms fall within the claimed scope of this invention. Also, since the invented method reveals the discovery, amenable for all types of processes and conversions, on which the author legitimately claims his intellectual ownership, be advised that applying the Index-K concept on any matter without the inventor's assent is unlawful. This is punishable by U.S. law, as well as the World International Intellectual Property Organization Treaty, and may result in severe civil and criminal penalties.

What I claim as my invention:

1. The extrapolation & modification of the U.S. Pat. No. 5,783,449 intended exclusively for quantifying alcohol catabolism, to extend the advanced tool for the quantification inner uniformity within biological processes/conversions by overstepping limitation for the prior method to compute a function of the cycle with non-zero baseline comprises steps of:
a. collecting the data of quantitative changes from process/conversion e.g., strength, activity, concentration, intensity, rate of transformation etc. vs. duration for the preferred bio-cycle;
b. replacing by zero-reference value the lowest corresponding meanings of the ensuing chances, in the case where neither initial or final points have particular zero values,
c. separately extracting the Area Under the Curve (AUC) values for ascending and descending shoulders of the plotted by this rule curve;
d. dividing the extracted descending AUC by ascending AUC meanings to obtain the advanced Index-K value.

2. The invention as claimed in claim 1 is the advanced Index-K quantification tool, which value reflects a distinctive inner quality of the selected process/conversion, and possesses balanced universal commensurability property since it represents the quotient of reacting vs. acting vital forces working by natural order of things in opposed directions in effort to conclude biological cycle.

3. A value of the advanced Index-K tool does not demonstrate direct ties to the technique, method, technology or unit of measurements, and depends commonly upon an inner regularity of the living cycle and correct measurement procedure.
